(19) United States
(12) Patent Application Publication
(10) Pub. No.: US 2022/0362577 A1
(43) Pub. Date: Nov. 17, 2022

(54) METHODS FOR GUIDING DIRECT DELIVERY OF DRUGS AND/OR ENERGY TO LESIONS USING COMPUTATIONAL MODELING

(71) Applicant: University of Vermont and State Agricultural College, Burlington, VT (US)

(72) Inventors: C. Matthew KINSEY, Williston, VT (US); Jason H.T. BATES, S. Burlington, VT (US)

(21) Appl. No.: 17/621,200

(22) PCT Filed: Jun. 22, 2020
(86) PCT No.: PCT/US2020/039029

§ 371 Date: Dec. 20, 2021

Related U.S. Application Data
(60) Provisional application No. 62/864,308, filed on Jun. 20, 2019.

(51) Int. Cl. A61N 5/10; G16H 50/50

(52) U.S. Cl. CPC A61N 5/1039; G16H 50/50; A61B 5/055

(57) ABSTRACT

A method for treatment of a tumor includes obtaining 3D imaging of the tumor; processing the 3D imaging of the tumor to obtain tumor morphology; determining a number of treatment sites, the locations of such sites, and the treatment dosage using a model of intratumoral treatment dynamics between vascular, intracellular, and extracellular space in order for the tumor to receive a therapeutic dosage at every location of the tumor; and treating the tumor at each of the determined treatment sites and with the determined treatment dosage. In some embodiments, the method further includes generating the model to include a plurality of interconnected volumes wherein each volume has one or more adjacent volumes with a shared boundary. One or more simulations of treatment over time may be conducted using the model, each simulation having a set of one or more initial parameters.

### CROSS-REFERENCE TO RELATED APPLICATIONS

This application claims priority to U.S. Provisional Application No. 62/864,308, filed on Jun. 20, 2020, now pending, the disclosure of which is incorporated herein by reference.

### FIELD OF THE DISCLOSURE

The present disclosure relates to guiding the application of therapy to a region of interest.

### BACKGROUND OF THE DISCLOSURE

Cisplatin is widely used to treat lung cancer because of its ability to interfere with cell replication. The usual route of administration is intravenous (IV). This exposes normal cells throughout the body to the toxicity of the drug, resulting in a heavy side-effect burden in off-target tissues. Moreover, cisplatin is taken up more readily by normal cells compared to tumor. For this reason, we and others have recently begun to explore the therapeutic potential of directly injecting cisplatin into lung tumors that are adjacent to airways accessible by bronchoscopy using an approach known as endobronchial ultrasound-guided transbronchial needle injection (EBUS-TBNI). This presumably allows higher drug concentrations to be achieved within the tumor while at the same time reducing both systemic concentrations and side effects. However, there is currently little data to guide the choice of injection site(s) within the tumor, or the dose per site. This leaves treatment planning for direct cisplatin injection into lung tumors on an entirely empirical footing, along with its attendant risks and missed opportunities.

### BRIEF SUMMARY OF THE DISCLOSURE

The present disclosure provides a method of treating tumors using a computational model. The computational model serves as a tool to guide decision making when performing intralesional therapies including injection of a drug (e.g., chemotherapy, immunotherapy, etc.) and energy application (e.g., radiofrequency and microwave ablation, etc.) for lesions such as tumors (e.g., lung lesions, etc.) The model makes predictions of the advantageous location(s), means of delivery (e.g., type of needle), delivery rate/time, and/or dose/energy for delivery of a chemical or energy therapy based on characteristics such as tissue and blood vessel density of the lesion and surrounding tissue structures (e.g., lung, etc.), and blood and tissue biopsy information. Embodiments may incorporate patient specific data providing a platform for personalized therapy

The present disclosure provides a method for guiding drug delivery to lesions using modeling, including an exemplary computational model of cisplatin pharmacodynamics following EBUS-TBNI. The model accounts for diffusion of cisplatin within and between the intracellular and extracellular spaces of a tumor, as well as clearance of cisplatin from the tumor via the vasculature and clearance from the body via the kidneys. We matched the tumor model geometry to that determined from a thoracic CT scan of a patient with lung cancer. The model was calibrated by fitting its predictions of cisplatin blood concentration versus time to measurements made up to 2 hours following EBUS-TBNI of cisplatin into the patient's lung tumor. This gave a value for the systemic volume of distribution for cisplatin of 12.2 L and a rate constant of clearance from the tumor into the systemic compartment of 1.46×10⁻⁴ s⁻¹. An embodiment of the presently-disclosed model indicated that the minimal dose required to kill all cancerous cells in a lung tumor can be reduced by roughly three orders of magnitude if the cisplatin is apportioned between five advantageously spaced locations throughout the tumor rather than given as a single bolus to the tumor center. Our findings suggest that optimizing the number and location of EBUS-TBNI sites has a dramatic effect on the dose of cisplatin required for efficacious treatment of lung cancer.

### DESCRIPTION OF THE DRAWINGS

For a fuller understanding of the nature and objects of the disclosure, reference should be made to the following detailed description taken in conjunction with the accompanying drawings, in which:

FIG. 1. Model structure. Each labeled rectangle represents a single well-mixed compartment.

FIG. 2. Measured blood concentration of cisplatin (open circles) following five 8-mg intratumoral injections into the lung tumor of a patient. Also shown (solid line) is the fit of Eq. 14 to the data (r²=0.98).

FIG. 3. (A) Axial CT image demonstrating a right paratracheal lung cancer, occurring in the prior radiation field. (B) Three-dimensional reconstructions of the tumor showing advantageous locations for 1 to 6 injections of cisplatin.

FIG. 4. Minimum total cisplatin dose needed for all tumor cells to reach a threshold intracellular concentration of 0.5×10⁻⁷ mg/mL as a function of the number of injections. Equal doses are given with each injection, and the injection sites are located advantageously.

FIG. 5. Percentage of killed carcinogenic cells as a function of threshold lethal concentration when a dose of 40 mg cisplatin is equally apportioned between 1 to 6 injections given at advantageous locations in the tumor.

FIG. 6. Sensitivity analysis of the model to variations in the three key parameters D, kᵢ, and k_{f}. For 1 (left) and 5 (right) advantageously-located injections, each parameter was adjusted in turn by +10% (white bars) and −10% (black bars). With each adjustment the percentage change in the lethal threshold concentration of cisplatin that would result in the death of all tumor cells with a total injected dose of 40 mg was determined.

FIG. 7. A chart depicting a method according to an embodiment of the present disclosure.

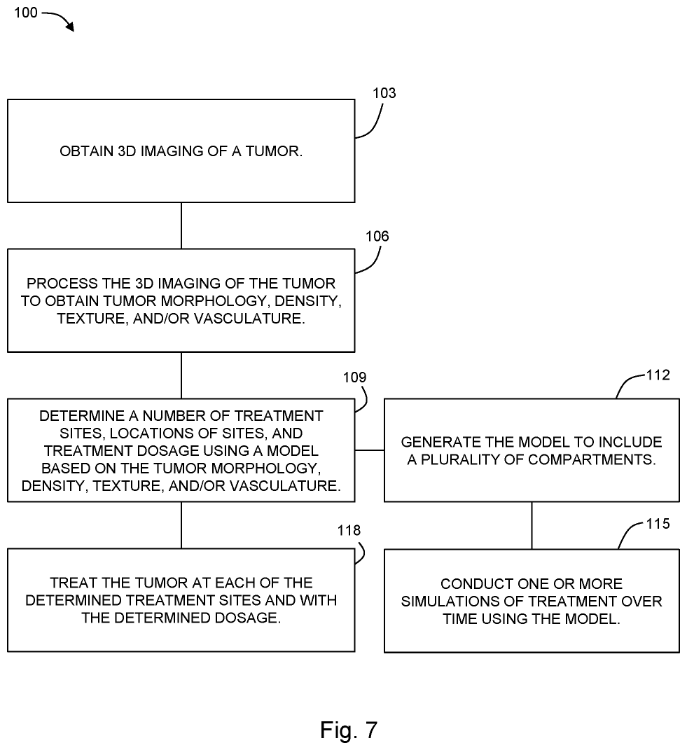

### DETAILED DESCRIPTION OF THE DISCLOSURE

No guidance exists for intralesional therapy with either a drug or energy in the lung. Intratumoral therapy for lung cancer is currently being performed in an ad hoc manner. For example, one center may apply an entire dose of drug to a single location, while another center apportions the total dose between a number of different locations-both approaches are entirely empirical. Neither approach uses an injection strategy that is guided by formal considerations of any tumor characteristics, including size. Similarly, thermal ablation therapy for lung tumors is currently applied using a standard thermal energy profile with little or no adjustment for the presence of regional heat sinks such as large blood vessels. Embodiments of the presently-disclosed model integrate lesion-specific tissue information derived from imaging to provide a rational basis upon which to make decisions about drug and/or energy delivery strategies in order to maximize therapeutic effectiveness while minimizing systemic side effects.

In an aspect, the present disclosure may be described as a method 100 for treatment of a tumor. The method may be embodied as a method for treating a tumor using a drug treatment such as, for example, chemotherapy, immunotherapy, etc. In some embodiments, the method may be embodied as a method for treating a tumor using a thermal treatment such as, for example, radiofrequency, microwave ablation, etc. The method 100 includes obtaining 103 three-dimensional (3D) imaging of the tumor. The 3D imaging may be radiographic, such as, for example, x-ray computed tomography (CT); magnetic-resonance imaging (MRI); ultrasound; and/or any other modality or combination of modalities to provide 3D imaging of a region of interest (ROI) of an individual. It should be noted that the term 3D image is used herein to describe data that can be used to generate 3D imaging information. For example, the 3D image may be a set of 2D images, such as, for example, a set of image slices. In some embodiments, the image is obtained 103 by retrieval from an electronic storage device. For example, the electronic storage device may be a disk drive, a flash drive, an optical drive, or any other type of memory. Such an electronic storage device may be local or remote (e.g., having an intervening network).

The obtained 103 3D imaging is processed 106 to obtain at least tumor morphology. In some embodiments, the 3D image may be processed to further obtain one or more of the density, the texture, and/or the vascularity of the tumor (e.g., for the entirety of the tumor, across the tumor, at multiple positions of the tumor, etc.)

The method includes determining 109 a number of treatment sites, the locations of such treatment sites, and the treatment dosage using a model of intratumoral treatment dynamics. The model is based on intratumoral dynamics of the treatment between vascular, intracellular, and extracellular space. For example, where the treatment is a drug therapy, the model is of the drug dynamics through the tumor. In another example, the treatment may be a thermal treatment, and the model may describe intratumoral thermal dynamics. The model is used to determine treatment such that a therapeutic dosage reaches each portion of the tumor.

In some embodiments, the number of treatment sites, locations of the treatment sites, and treatment dosage is determined 109 by generating 112 the model and conducting 115 one or more simulations of treatment over time using the generated model. The model may comprise a plurality of interconnected compartments representing the tumor morphology. In embodiments of the present disclosure, the model of a tumor may be mapped to the specific anatomy of a tumor in a patient as determined by imaging. In some embodiments, the method 100 may include treating 118 the tumor at each of the determined treatment sites and with the determined treatment dosage.

The tumor is modeled as a plurality of volumes. In an example, a tumor may be modeled where its morphology is divided into a plurality of cuboid volumes. Each of the volumes may be of equal size (e.g., volume, dimensions, etc.) as the other volumes or may be otherwise sized. The number of volumes (and thus size of each volume) may be pre-determined. In some embodiments, greater numbers of smaller volumes may provide more accurate modeling, while requiring additional computational power. Each volume may represent a topographically coincident, but functionally distinct compartments. For example, the model may be such that each volume includes intracellular space and extracellular space. In another example, the model may include compartments of intracellular space, extracellular space, and vasculature.

Treatment dynamics (direction and magnitude) through the tumor may be modeled based on the volumes and compartment types as further detailed below. The model may have initial conditions (parameters) representing, for example, the location and amount of drug immediately following its injection into the tumor.

In embodiments wherein multiple simulations are conducted, each simulation may be conducted by setting a set of one or more initial parameters. Such initial parameters may include, for example, one or more treatment location(s), a delivery modality (e.g., size and/or type of needle, etc.), a delivery rate, a delivery dose, tissue diffusivity, tissue perfusion, and/or other parameters. Where more than one treatment location is provided, other parameters may have a value for each treatment location. For example, a delivery dose may be provided for each treatment location, and each provided delivery dose may be the same as or different from the other delivery dose(s). The simulation is allowed to run by calculating the therapy's movement between each pair of adjacent volumes (for example, magnitude and direction) over time using the applicable classification model for each compartment and adjacent compartments. In this way, the model may show, for example, for a given load and location of therapy (e.g., drug, heat, etc.) within the tumor, a therapy's effect (e.g., drug concentration, thermal change, etc.) within each compartment over time, etc. Each simulation may be run with differing initial parameters. In this way, the resulting outcomes of each simulation may be evaluated.

The features of the ROI that may be incorporated into the model include not only the surface corresponding to its boundary but also any other anatomic or biophysical features that can be discerned in the image or determined from analysis of biopsy specimens. These other features include topographic distributions of tissue density and perfusion, as well as vascular anatomy. The model may then be used to simulate how both diffusion and convection cause an injected agent (either drug or energy) to distribute throughout the ROI and to eventually become cleared from it as a function of time. Interrogating these simulations allows the determination of patient-specific injection strategies (i.e., sets of injection locations together with dose per location) that achieve an optimal (advantageous) balance between maximizing the therapeutic effect of the agent or energy within the ROI while minimizing the total delivered dose. The total dose for pharmaceuticals is simply the total amount of delivered drug, while for energy-based therapies the total dose is given by the product of power and delivery time. The model may thus provide personalized therapeutic guidance for treatment of, for example, lung lesions.

Some embodiments comprise a software package that interfaces with imaging and tissue biopsy data allowing users to create a lesion-specific model. Systematic search algorithms may then interrogate the model in order to identify advantageous treatment strategies regarding dose and location of delivery. Embodiments of the software will also be able to interface with intraoperative imaging modalities such as, for example, cone-beam CT and ultrasound. This will provide real-time guidance for the placement of the drug and/or energy delivery device into a lung lesion at advantageous locations as determined by the computational model.

An anatomically-based mathematical/computational model of cisplatin dynamics within a peribronchial lung tumor may be useful in the design of therapeutic injection strategies, and may address the question of the optimal number of injections to be performed. Such a model is developed in this disclosure, and the model is applied to a high-resolution computed tomography (CT) scan of a patient's lung tumor. We show that by accounting for intratumoral diffusion of drug between extra-and intracellular compartments, as well as its convective clearance via the vasculature, we can move toward a rationale design for direct lung cancer treatment strategies using EBUS-TBNI of cisplatin.

In another aspect, the present disclosure may be embodied as a system for treatment of a tumor. The system includes a communication interface and a processor in communication with the communication interface. The system may further include a storage device, such as a hard disk, flash drive, etc. attached directly or indirectly (e.g., by way of a network, etc.) to the communication interface. The processor is programmed to perform any of the presently-disclosed methods. For example, the processor may be programmed to: obtain 3D imaging of the tumor from the communication interface; process the 3D imaging of the tumor to obtain tumor morphology; determine a number of treatment sites, the locations of such sites, and the treatment dosage using a model of intratumoral treatment dynamics between vascular, intracellular, and extracellular space in order for the tumor to receive a therapeutic dosage at every location of the tumor; and provide, to a user, a treatment plan for treating the tumor at each of the determined treatment sites and with the determined treatment dosage. For example, the treatment plan may be provided by displaying the plan on a display. In some embodiments, the treatment plan may be provided by e-mail, or other communication modality. In some embodiments, the processor is further programmed to provide treatment instructions to an output interface and/or the communication interface. For example, the output interface may be connected to an automatic means for applying the therapy, which is configured to receive the instructions and apply the appropriate therapy (e.g., inject a drug therapy, apply a thermal therapy, etc.) to the tumor.

In another aspect, the present disclosure may be embodied as a non-transitory computer-readable medium having stored thereon a computer program for instructing a computer to perform any of the presently-disclosed methods. For example, a non-transitory computer-readable medium may include a computer program to obtain 3D imaging of a tumor from a communication interface; process the 3D imaging of the tumor to obtain tumor morphology; determine a number of treatment sites, the locations of such sites, and the treatment dosage using a model of intratumoral treatment dynamics between vascular, intracellular, and extracellular space in order for the tumor to receive a therapeutic dosage at every location of the tumor; and provide to an output interface, a treatment plan for treating the tumor at each of the determined treatment sites and with the determined treatment dosage.

### Example 1—Drug-Based Therapies

An exemplary model suitable for use in modeling drug therapies is provided with discussion of Cisplatin used for a lung tumor. It should be noted that this is an example (i.e., non-limiting) and is used to illustrate the broader use of models for drug therapies.

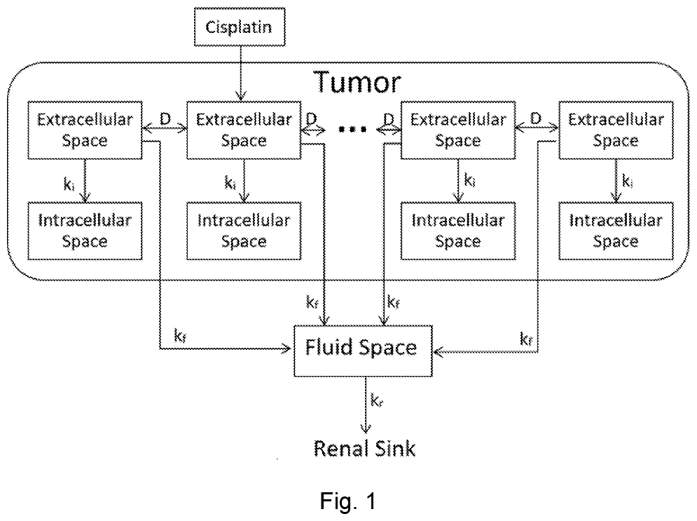

Model development. In an exemplary model, a tumor is represented as a superposition of two distinct spaces, the extracellular space and the intracellular space, both of which are assumed to have volumes that do not change over the timescale of the model. The extracellular space comprises the interstitial fluid and connective tissue within a tumor, while the intracellular space comprises the cytoplasm and associated organelles, including the nuclei, of the malignant cells. The small blood vessels that perfuse the tumor are contiguous with, and thus part of, a separate fluid space that includes the systemic vasculature and possibly also some extravascular spaces of distribution within the tissues of the body. Cisplatin is eventually excreted from the body, predominately via the kidneys. For the purposes of developing a continuous mathematical theory of the model, the extracellular and intracellular spaces are assumed to comprise, within each infinitesimal volume of tumor, two topographically coincident but functionally distinct compartments occupying volume fractions of αₑ and αᵢ, respectively, where αₑ+αᵢ=1. The model thus includes a series of interconnected pairs of extracellular and intracellular compartments that link to a single fluid compartment as illustrated in FIG. 1.

When cisplatin is first injected transbronchially into a tumor it enters the extracellular space at the site of injection from which it proceeds to diffuse throughout the rest of the extracellular space with diffusion coefficient D. The cisplatin subsequently leaves the extracellular space by flowing into the intracellular and fluid spaces within the tumor with rate-constants per unit volume of kᵢ and k_{f}, respectively. In the intracellular space it binds immediately and irreversibly to the cell DNA, so the intracellular space is modeled to act as a sink for cisplatin. When cisplatin passes into the blood vessels it is rapidly convected into the rest of the fluid space where it mixes with the blood over a time-scale of minutes, which is short compared to the time-scale of the model. Thus, the fluid space is considered to act as a single well-mixed compartment. Cisplatin diffuses from the fluid space back into the extracellular space with rate constant k_{f}′.

Based on the above considerations, the concentration of cisplatin in the extracellular space, φₑ(\vec{r}, t), is governed by the equation:

$$\frac{d {φ}_{e} ( {r}^{⟶} , t )}{dt} = D {▽}^{2} {φ}_{e} ( {r}^{⟶} , t ) - ( {k}_{i} + {k}_{f} ) {φ}_{e} ( {r}^{⟶} , t ) + {k}_{f}^{′} {φ}_{f} ( t ) , \qquad ( 1 )$$

where φ_{f}(t) is the concentration of cisplatin in the fluid space. The fluid space constitutes a single well-mixed compartment so φ_{f}(t) is a function purely of time, and is governed by:

$$\frac{d {φ}_{f} ( t )}{dt} = \frac{1}{{V}_{f}} \frac{d [ {\int }_{V} {k}_{f} {φ}_{e} ( \vec{r} , t ) d \vec{r} ]}{dt} - {k}_{f}^{′} {φ}_{f} ( t ) - {k}_{r} {φ}_{f} ( t ) , \qquad ( 2 )$$

where kᵣ is the rate-constant for renal excretion and V_{f} is the total fluid volume. Finally, cisplatin accumulates locally in the intracellular space from its adjacent extracellular supply, so the intracellular concentration, φᵢ(\vec{r}, t), is governed simply by:

$$\frac{d {φ}_{i} ( \vec{r} , t ) \to }{dt} = {k}_{i} {φ}_{e} ( \vec{r} , t ) . \qquad ( 3 )$$

When cisplatin is injected directly into a tumor, the above equations can be simplified by noting that φ_{f}(t) is always much lower than the early values of φₑ(\vec{r}, t), which means that these early values of φₑ(\vec{r}, t) are chiefly responsible for generating the clinically effective concentrations of cisplatin in the intracellular space. This allows for approximating the fluid space as a sink for cisplatin, which reduces Eq. 1 to:

$$\frac{d {φ}_{e} ( \vec{r} , t )}{dt} = D {∇}^{2} {φ}_{e} ( \vec{r} , t ) - ( {k}_{i} + {k}_{f} ) {φ}_{e} ( \vec{r} , t ) . \qquad ( 4 )$$

Let a dose M of cisplatin be injected at a location in the tumor centered on point \vec{r}ᵢ within the extracellular space at t=0 such that approximate φₑ(\vec{rₗ},0) can be approximated as Mδ(\vec{rₗ}), where δ is the Dirac delta-function. The solution to Eq. 4, assuming the tumor boundary to be at infinity, is then:

$${φ}_{e} ( \vec{r} , t ) = \frac{M {e}^{- ( {k}_{i} + {k}_{f} ) t}}{\sqrt{4 {π ( Dt )}^{3}}} {e}^{\frac{- {\vert \vec{r} - \vec{{r}_{j}} \vert}^{2}}{4 D t}} . \qquad ( 5 )$$

If M is distributed between N different injection locations within the tumor, the superposition principle gives:

$${φ}_{e} ( \vec{r} , t ) = {\sum }_{j = 1}^{N} \frac{{m}_{j} {e}^{- ( {k}_{i} + {k}_{f} ) t}}{\sqrt{4 {π ( D t )}^{3}}} {e}^{\frac{- {\vert \vec{r} - \vec{{r}_{j}} \vert}^{2}}{4 D t}} \qquad ( 6 )$$

where M=Σⱼ₌₁^{N}m.

From Equations 3 and 6, we then have:

$$\begin{matrix}{φ}_{i} ( \vec{r} , t ) = {k}_{i} {\int }_{0}^{t} {φ}_{e} ( \vec{r} , τ ) d τ \\ = {k}_{i} {\sum }_{j = 1}^{N} \frac{{m}_{j}}{2 D \vert \vec{r} - \vec{{r}_{j}} \vert} [ {e}^{- 2 αβ} ( 1 - erf ( α - β ) ) \\ - {e}^{- 2 αβ} ( 1 - erf ( α + β ) ) ]\end{matrix} \qquad ( 7 )$$

$$where$$

$$α = \sqrt{\frac{{\vert \vec{r} - \vec{{r}_{j}} \vert}^{2}}{4 D τ}} and β = \sqrt{( {k}_{i} + {k}_{f} ) τ} . \qquad ( 8 )$$

and erf(α+β) is the error function.

The ability of cisplatin to eradicate cancer depends in some way on its intracellular concentration profile, but exactly how remains a matter of debate. In the interests of avoiding unnecessary complexity, the present model may be guided by the fact that in the cytoplasm the cis chlorine groups in the cisplatin molecule are replaced by water molecules, allowing it to bind essentially irreversibly to DNA. This interferes with the ability of DNA both to replicate and to repair itself, eventually leading to cell death by apoptosis. Cytotoxicity is thus clearly related to the mass accumulation of cisplatin within the nucleus, which under the assumptions described above can be approximated by its asymptotic intracellular concentration φᵢ(\vec{r}, t→∞).

Model fitting. We assume that cisplatin does not leave the tumor at its boundary, since there is very little tissue beyond the boundary for it to move into, so the boundary may affect the shape of φₑ(\vec{r}, t). However, if we assume that the boundary essentially reflects back into the tumor any drug that would have otherwise diffused beyond it, the total amount of cisplatin remaining in the extracellular space will remain relatively unaffected, in which case we can estimate this total amount by spatially integrating Eq. 6 to infinity to obtain:

$${M}_{e} ( t ) = {\int }_{V} {φ}_{e} ( \vec{r} , t ) dV = {\sum }_{j = 1}^{n} {m}_{j} {e}^{- ( {k}_{i} + {k}_{f} ) t} . \qquad ( 9 )$$

Similarly, substituting Eq. 6 into Eq. 3 and spatially integrating the result to infinity gives:

$${M}_{i} ( t ) = {\sum }_{j = 1}^{N} \frac{{α}_{i}}{{α}_{e}} \frac{{k}_{i}}{{k}_{i} + {k}_{f}} {m}_{j} ( 1 - {e}^{- ( {k}_{i} + {k}_{f} ) t} ) . \qquad ( 10 )$$

The amount of cisplatin that has moved from the extracellular space to the fluid space at any point in time is simply the initially injected amount less the amounts in the extracellular space (Eq. 9) and the intracellular space (Eq. 10). That is, from Eq. 2:

$${\int }_{V} {k}_{f} {φ}_{e} ( \vec{r} , t ) d \vec{r} = {\sum }_{j = 1}^{N} ( 1 - \frac{{α}_{i}}{{α}_{e}} \frac{{k}_{i}}{{k}_{i} + {k}_{f}} ) {m}_{j} ( 1 - {e}^{- ( {k}_{i} + {k}_{f} ) t} ) . \qquad ( 11 )$$

Finally, we assume that the intracellular space is much smaller than the extracellular space (i.e., αₑ>>αᵢ), so Eq. 11 becomes:

$${\int }_{V} {k}_{f} {φ}_{e} ( \vec{r} , t ) d \vec{r} = {\sum }_{j = 1}^{n} {m}_{j} ( 1 - {e}^{- ( {k}_{i} + {k}_{f} ) t} ) . \qquad ( 12 )$$

Fluid cisplatin concentration, φ_{f}(t), is the result of a balance between the flow of drug from the extracellular to the fluid space and the drug clearance rate due to blood filtration by the kidneys, which will be modeled as a sink with time constant kᵣ. Combining Eqs. 2 and 12 gives:

$$\frac{d {φ}_{f} ( t )}{dt} = \frac{1}{{V}_{f}} \frac{\partial }{\partial t} ( {\sum }_{j = 1}^{n} {m}_{j} ( 1 - {e}^{- ( {k}_{i} + {k}_{f} ) t} ) ) - {k}_{r} {φ}_{f} ( t ) \qquad ( 13 )$$

$$so$$

$${φ}_{f} ( t ) = {\sum }_{j = 1}^{n} \frac{{k}_{f} + {k}_{i}}{{k}_{f} + {k}_{i} - {k}_{r}} \frac{{m}_{j}}{{V}_{f}} ( {e}^{- k , t} - {e}^{- ( {k}_{i} + {k}_{f} ) t} ) . \qquad ( 14 )$$

Cisplatin biological half-life in humans has been reported to be approximately 30 min, so it is assigned the value kᵣ=3.85·10⁻⁴ s⁻¹.

Equation 14 was fit to the measured cisplatin blood concentrations obtained from the human subject. The fitting was achieved by optimizing the values of the two free parameters (k_{f}+kᵢ) and V_{f} using a gradient-based algorithm to minimize the cost function:

$${J}_{Blood} ( {k}_{f} + {k}_{i} , {k}_{f} ) = \sqrt{{\sum }_{N = 1}^{5} {[ {ϕ}_{{blood}_{data}} - {ϕ}_{{blood}_{model}} ]}^{2}} . \qquad ( 15 )$$

Patient data. We maintain an ongoing protocol, approved by the Institutional Review Board of the University of Vermont, to evaluate tissue and correlative data obtained during clinically indicated EBUS-TBNI of cisplatin. All patients referred for potential EBUS-TBNI of cisplatin are reviewed at the Multidisciplinary Lung Tumor Board of the University of Vermont Medical Center to insure that there are no other more well-established therapeutic options. Patients provided informed consent, and all research procedures were conducted in accordance with Good Clinical Practice (GCP) as outlined by the Collaborative Institutional Training Initiative (CITI).

In order to understand potential toxicities from EBUS-TBNI of cisplatin, serial cisplatin blood level monitoring was performed during and following a single procedure. This allowed us to determine when blood cisplatin levels peaked in order to guide the timing of blood draws in subsequent cases. Five 8-mg cisplatin injections were delivered into the tumor of a patient with recurrent lung cancer. Ultrasound guidance was used to attempt to distribute the 5 injections evenly throughout the tumor over an interval of 18 min. The concentration of cisplatin was measured in venous blood drawn at 5, 15, 30, 60 and 120 min after the final injection.

The patient also underwent a high-resolution computed tomography (CT) scan of the thorax from which the location, size and 3D shape of the lung tumor was accurately determined. We used MATLAB 2015b (The MathWorks, Natick, Mass., USA) to create a geometrically accurate representation of the tumor boundary from the CT image.

Optimizing injection strategies. We assume that tumor cell death occurs when the intracellular concentration of cisplatin reaches a lethal threshold level of (Pt. There is little guidance in the literature as to the appropriate value of Pt to use in the model, so for our initial simulations we arbitrarily chose a nominal value of 0.5 mg/mL. This is a relatively conservative estimate since it implies that at least half of the delivered agent (20 of the injected 40 mg distributed throughout a 40 ml tumor) must be absorbed into the cell nucleus to be cytotoxic, which will happen in any cell in which φₜ is exceeded by the asymptotic value of φₜ(\vec{r}, t) given by Eq. 7. This asymptotic value is:

$${φ}_{i} ( \vec{r} , t \to ∞ ) = {\sum }_{j = 1}^{N} \frac{{k}_{i} {m}_{j}}{D \vert \vec{r} - \vec{{r}_{j}} \vert} \cdot {e}^{- 2 αβ} \qquad ( 16 )$$

The value of D for a drug in normal tissues at 37° C. is reported to be a function of the drug molecular weight according to the empirical law:

D=1.778·10⁻⁴(MW)−^{0.75} cm²/s. (17)

The molecular weight of cisplatin is 300 Da, giving a value of D of 2.47×10⁻⁶ cm² s⁻¹.

For EBUS-TBNI to successfully treat a lung tumor, φᵢ(\vec{r}, t→∞) must exceed φ(t) everywhere within the tumor. Achieving this condition depends on both the total cisplatin dose, M, and the manner in which this dose is apportioned between different injection sites within the tumor. At the same time, it is clearly to the patient's benefit to have M be as small as possible so that systemic side effects are minimized. Accordingly, based on the model of cisplatin dynamics developed above, we determined the locations and doses of N injections that would minimize M subject to the condition φᵢ(\vec{r}, t→∞)>φₜ at every point within the tumor, for N=1, . . . , 6. We identified these optimum injection strategies by first using a genetic algorithm to determine the spatial locations of N injections that maximized the minimum value of φᵢ(\vec{r}, t→∞) within the tumor for specified values of N and M. Since the model predictions scale linearly with M, finding the minimum value of M was then simply a matter of scaling the doses so that the minimum value of φᵢ(\vec{r}, t→∞) equaled φₜ.

### Results

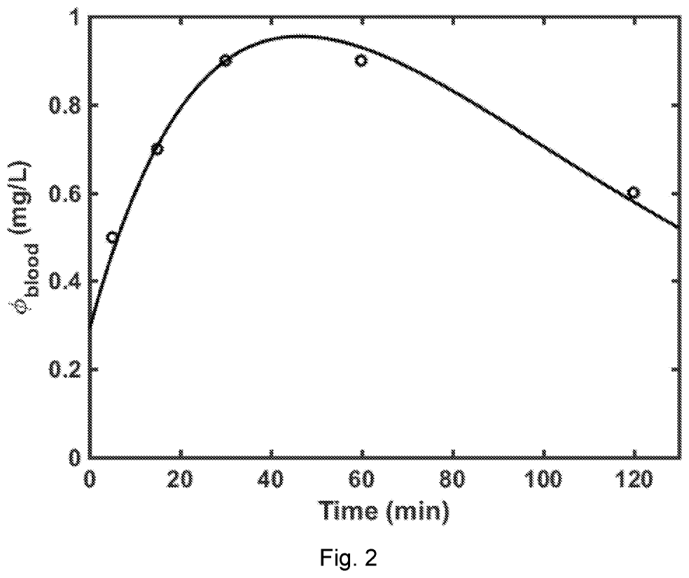

The fit of Eq. 14 to the data of cisplatin blood concentration versus time after injection is shown in FIG. 2, and yields values for the two independent parameters of V_{f}=12.2 L and kᵢ+k_{f}=2.51×10⁻⁴ s⁻¹. Previous studies in head, neck and gastric carcinomas have reported kₗ=1.05×10⁻⁴ s⁻¹, so if we assume the same to be true for lung carcinomas then we obtain k_{f}=1.46×10⁻⁴ s⁻¹.

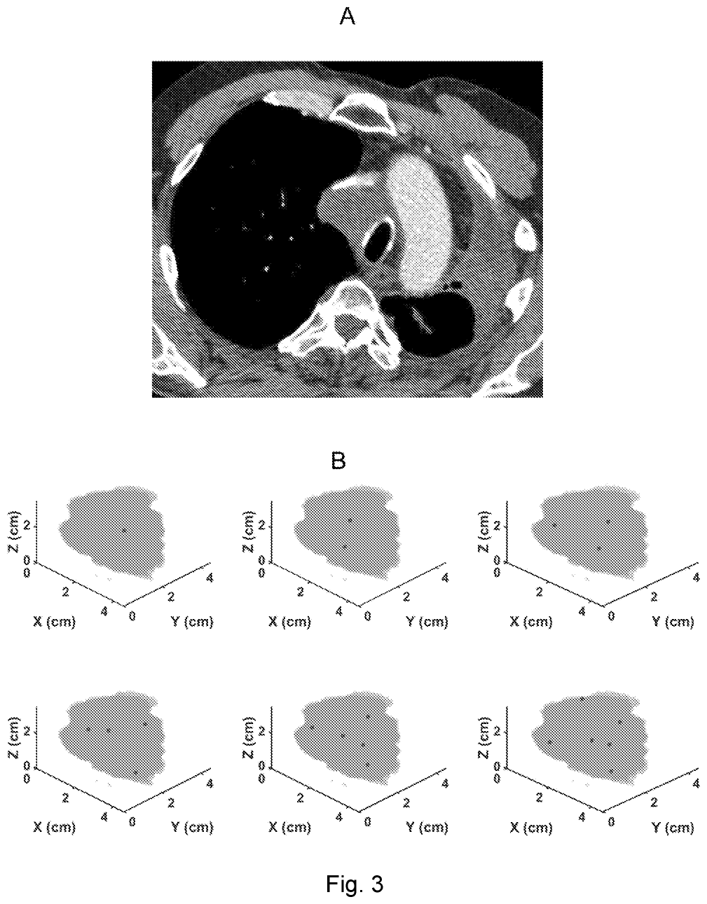

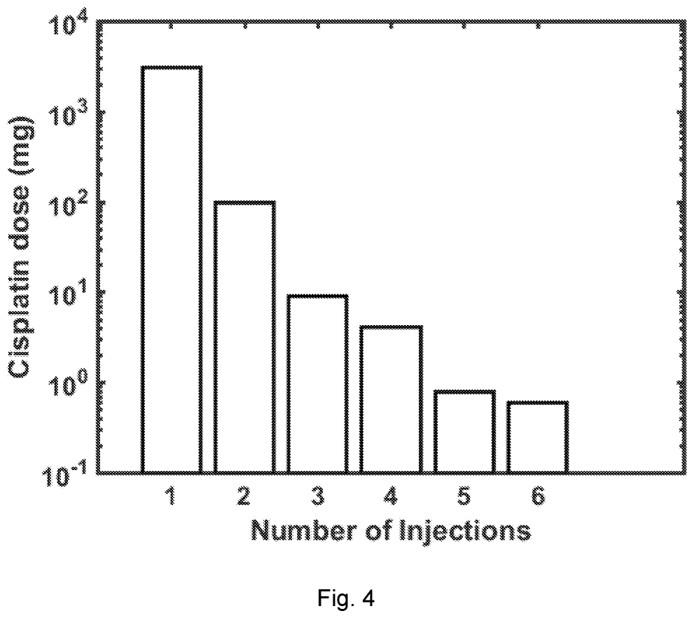

FIG. 3 shows renditions of the lung tumor in the patient we studied along with advantageous locations of cisplatin injection sites for 1 to 6 injections calculated using the computational model with the values of V_{f}, kᵢ, and k_{f} given above. Note that a single injection is optimally located close to the middle of the tumor while multiple injections are distributed in a balanced way throughout the tumor mass, as one would expect intuitively. The major benefit of multiple injections, however, is evident in FIG. 4 which shows the total cisplatin dose required to kill all tumor cells as a function of the number of injections. This dose decreases by more than 3 orders of magnitude in going from 1 to 5 injections. Relatively little additional dose reduction is achieved by going to 6 injections, however.

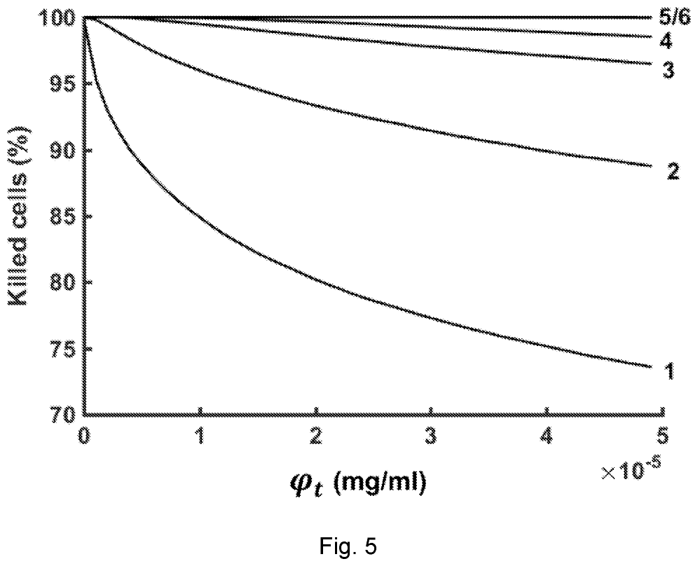

Another aspect of the benefits of multiple advantageously-placed cisplatin injections is revealed in FIG. 5 which shows the predicted fraction of tumor cells that would be killed as a function of the lethal threshold concentration φₜ following a total cisplatin dose of 40 mg of cisplatin, which is the dose currently being used for EBUS-TBNI clinically. A single injection of cisplatin at this dose fails to kill all tumor cells at the lowest value of φₜ=10⁻⁷ mg/mL, and by φₜ=5×10⁻⁷ mg/mL less than 75% of the tumor has been eradicated. Indeed, complete cell killing is not achieved with a single injection until φₜ falls to 6.54×10⁻⁹ mg/mL. In contrast, 5 injections are completely effective until φₜ=2.73×10⁻⁵.

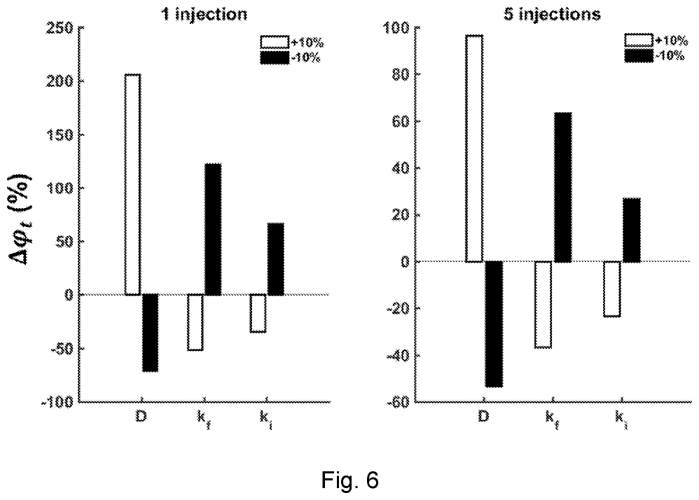

A sensitivity analysis of the model predictions to the values of the parameters D, k_{f}, and kᵢ was performed by adjusting each parameter in turn by ±10% of its best-fit value and then determining how this affected the maximum value of φₜ at which complete tumor killing was achieved with a total cisplatin dose of 40 mg. FIG. 6 shows these calculations for 1 and 5 injections. The parameter sensitivities are substantially less for 5 injections than for a single injection, again speaking to the relative advantages of the multiple injection strategy. This analysis also shows that increasing the rate of diffusion within the intracellular space raises φₜ and thus permits complete killing with a reduced dose of cisplatin. This is not surprising since more rapid spread of cisplatin to sites within the tumor that are distant from the sites of injection will allow concentrations at the distant sites to rise to higher levels before the drug is cleared. Conversely, increasing either k_{f} or kᵢ causes the value of φₜ to decrease, presumably because increasing the loss of drug to sinks near the injection sites reduces the amount left to diffuse to distant parts of the tumor.

### Discussion

EBUS-TBNI of cisplatin has recently emerged as an alternative treatment for peribronchial lung tumors, in order to achieve high intratumoral concentrations while reducing harmful off-target side effects. There is currently no consensus as to how cisplatin should be delivered in to a tumor, nor is it known how injection strategy impacts treatment efficacy. It nevertheless seems reasonable to suppose that efficacy should depend on the number, location and dose of individual injections. Determining the optimal injection strategy for a given tumor is, however, a very non-trivial task given the number of disparate factors that come into play. These factors include tumor volume and shape, the nature of the perfusing vasculature, and features of the tumor tissue including its density and the orientation of fascial planes. While not all of these can be determined in a noninvasive fashion, tumor shape is accurately resolved in a CT scan. We exploited this opportunity in the present study to investigate how cisplatin might distribute itself throughout the tumor from a number of specified injection sites, albeit in a model that approximates reality in numerous ways not the least of which is the assumption that the tumor tissue is biophysically homogeneous and isotropic. Nevertheless, this simple model provides an accurate accounting of cisplatin in the blood as a function of time following EBUS-TBNI of cisplatin into the tumor of a patient with lung cancer (FIG. 2). The model yields a value for the extra-tumoral volume of distribution (V_{f}) of 12.2 L, which is similar to volume of the extracellular fluid compartment in a 70 kg adult man; this volume is known to have a value in L that is approximately 20% of body weight in kg. The model also yields a value for k_{f} that is similar to published values of kᵢ, which is not unexpected given that these two rate constants reflect rates of diffusion between different compartments of the same tumor tissue. The presently-disclosed model thus appears to capture the overall nature of cisplatin kinetics within the body and consequently has the potential, at the very least, to help quantify systemic exposure to this noxious drug.

A finding of the present study, however, is the enormous apparent benefit of apportioning a given dose of cisplatin between a number of well-placed injections rather than delivering the entire dose into a single central location, as shown in FIG. 3. Indeed, the presently-disclosed model predicts that the dose of cisplatin required to kill a given fraction of tumor cells using five injections can be 3 orders of magnitude less than that required for a single injection (FIG. 4). At six injections we appear to be approaching the point of diminishing returns, but these results provide compelling evidence that EBUS-TBNI should not be limited to a single injection site in the treatment of lung cancer. This conclusion is further supported by the results shown in FIG. 5 which indicate that increasing the number of injections has a marked effect on the robustness of treatment efficacy in the presence of variations in the local lethal concentration of cisplatin; five or more injections are predicted to be almost uniformly efficacious over the range of Pt studied while a single injection is relatively fragile in this respect. Of course, these results are predicated on the cisplatin injections being delivered at the advantageous sites predicted by our model. On the other hand, the locations of these advantageous sites are distributed roughly uniformly throughout the body of the tumor (FIG. 3). It may therefore be that empirical placement of injections guided simply by the principle of uniform distribution will be close enough to optimal that most of the predicted gains of multiple injections will be realized.

Certain parameters in the model were assigned values based on information from the literature, such as a diffusion coefficient reported in normal tissues and an intracellular rate-constant matched to values reported for neck and gastric tumors. There will always remain uncertainty in these values, not to mention the fact they may exhibit significant spatial variations within a given tumor. The parameter sensitivity analysis presented in FIG. 6 shows that our model predictions can be rather sensitive to errors and/or uncertainties in these parameters. Indeed, 10% variations in the parameter D can affect predictions of tumor killing by as much as 200% (FIG. 6). Thus, the predictions of some embodiments of the model may not provide precise guidelines as to the total dose of cisplatin to administer to any particular tumor. In some embodiments, a margin of safety, such as, for example, several fold above the predicted minimal dose, may be clinically advisable. Nevertheless, an increase of several-fold in the total dose given in five well-placed injections is still vastly less than the 3 orders of magnitude increased dose required in a single injection, underscoring the apparent importance of distributing the initial cisplatin load at multiple sites throughout the tumor.

In the present study, we chose a rather straightforward cytotoxicity function, namely that a cell dies when its total cisplatin load exceeds a specified lethal threshold. This, however, can easily be modified to some other concentration-lethality function should a better alternative come to light, and such alternatives are within the scope of the present disclosure.

We also made certain simplifying assumptions in deriving the model equations, such as the volume of the intracellular space being smaller than that of the extracellular space, and neglecting the return of drug to the tumor from the fluid space. These assumptions were made not only in the interests of arriving at analytic solutions to the model equations that are rapidly solvable, but also because cisplatin binds irreversibly to DNA. There nevertheless remains the possibility that the cytoplasmic cisplatin concentration could rise to the level where it starts to efflux out of the cell before having a chance to bind to the DNA. However, other research has found that cisplatin-treated cells demonstrated stepwise decrements in mitochondrial respiration with increasing concentrations of cisplatin above 5.0 uM, implying that this was below the concentration at which all binding sites are saturated. In the present study, 133 moles of cisplatin was injected into a tumor having a volume of roughly 40 ml, so even if every molecule of cisplatin was absorbed irreversibly by the tumor cells, we would reach a maximum concentration of 3.3 uM. Furthermore, the presently-disclosed model indicates that the rate-constants governing flux of cisplatin into the intracellular and vascular spaces from the extracellular space are roughly the same, so the maximum possible intracellular concentration would then be only half of this, or about 1.6 uM, and even this concentration would be achieved only transiently. Thus, it seems likely that the maximum intracellular dose achieved in the model would be well below that needed to saturate all cisplatin binding sites for the majority of the time following injection, making the cisplatin efflux from intracellular to extracellular spaces correspondingly small.

In conclusion, we have developed a mathematical/computational model of cisplatin pharmacodynamics that allows us to predict the distribution and ultimate fate of cisplatin delivered to a lung tumor via EBUS-TBNI. We used the model to predict the minimal efficacious dose of cisplatin and its optimal sites of administration in an accurately reconstructed tumor imaged in a patient with lung cancer. The model gave an accurate fit to measured concentrations of cisplatin in the blood over the 2 hours following injection. The model predicted that dramatic reductions in the effective dose of cisplatin in this tumor would be possible if the drug was apportioned between 5 appropriately selected sites throughout the tumor rather than being delivered in its entirety at a single central site.

### Example 2—Energy-Based Therapies

In another example, the model may be used to guide energy-based therapies. Such therapies that rely on the application of high frequency energy to destroy biological tissues have been in use for several decades in fields such as Cardiology and more recently, Interventional Radiology. This latter specialty has delivered both microwave and radiofrequency energy via catheters to induce thermal destruction of lesions in the lung. However, the distances involved in traversing the lung and chest wall have limited the precise application of such therapies. Further, the complicated heterogeneous structure of different lung lesions, together with the varying proximities of large blood vessels that serve as the primary heat sink, has left the therapy on an empiric footing. This example demonstrates how the above approach can be applied to energy-based therapies. The exemplary model described below incorporates patient-specific image data to estimate the optimal dose, timing and location of energy delivery to a lung lesion.

It is assumed that a lung lesion can be represented as a superposition of three distinct tissue spaces—extracellular space, intracellular space, and vascular space-between which energy transits not according to Fick's law (as for intratumoral drug delivery) but by the bioheat transfer equation. This energy balance equation is a standard model for predicting the temporal evolution of temperature distributions in tissues, and includes both diffusive and convective modes of heat transport:

$$ρ {c}_{p} \frac{\partial T}{\partial t} = ∇ \cdot κ ∇ T + {Q}_{p} + {Q}_{m} - {Wc}_{b} ( T - {T}_{b} ) \qquad ( 18 )$$

where ρ is the tissue mass density (kg·m⁻³), K is the thermal conductivity (W·m⁻¹·° C.⁻¹), T is the local tissue temperature (° C.), Qₚ is the local energy deposited by the therapeutic modality (J·m⁻³), and Qₘ is the metabolic heat generation (J·m⁻³). For the present purposes, we will ignore metabolic heat generation, given that it is orders of magnitude smaller than the heat required for tissue ablation. W is the blood perfusion per unit volume of tissue (kg·m⁻³·s⁻¹), C_{b} is the specific heat of blood (J·Kg⁻¹·° C.⁻¹), and T_{b} is blood temperature (° C.). As it stands, this equation accounts only for the magnitude of perfusion and not the direction of blood flow, but that can be rectified if necessary by replacing the term with a term proportional to the scalar product of the blood flow vector field and the gradient of the temperature scalar field.

Radiofrequency ablation occurs via Joule heating, in which case we assume that displacement currents within the tissue are negligible (microwave ablation, which we do not consider here, involves dielectric heating that is described by Maxwell's equations). The tissue is thus considered to be purely resistive, in which case the heat source density is given by:

q=JE (19)

where J is the current density (A·m⁻²), and E is the electric field intensity (V·m⁻¹).

Since the electric field energy delivered to the tissue by a point electrode falls off as the square root of the distance, r (mm), from the electrode, the bioheat equation becomes:

$$ρ {c}_{p} \frac{\partial T}{\partial t} = κ ( T , t ) ∇ \cdot ∇ {T}_{( r , t )} + \frac{σ P}{{r}^{4}} - P ∇ T ( r , t ) \qquad ( 20 )$$

where P is a measure of the electric field energy emanating from the electrode and a is the tissue conductance density (Ω⁻¹·mm⁻²).

The above equations are solved throughout a specific tumor geometry and tissue property distribution in order to determine the generation and subsequent dissipation of heat within the tumor. Heat dissipation is accounted for in the tissue volumes (compartments) as described in the drug diffusion model above, namely the extracellular space, the intracellular space, and the vascular space.

This approach provides the basis for determining the distribution of heat energy within a tumor, such as a lung tumor, or other form of lesion. Ablation energy can then be tuned in order to target a desired temperature profile within the tissue, such as reaching a specified minimum temperature at every tissue location of the lesion. This provides the ability to guide ablation treatment decisions in order to improve therapeutic efficacy while reducing side effects.

Although the present disclosure has been described with respect to one or more particular embodiments, it will be understood that other embodiments of the present disclosure may be made without departing from the spirit and scope of the present disclosure.

What is claimed is:

1. A method for treatment of a tumor, comprising:
obtaining 3D imaging of the tumor;
processing the 3D imaging of the tumor to obtain tumor morphology;
determining a number of treatment sites, the locations of such sites, and the treatment dosage using a model of intratumoral treatment dynamics between vascular, intracellular, and extracellular space in order for the tumor to receive a therapeutic dosage at every location of the tumor; and
treating the tumor at each of the determined treatment sites and with the determined treatment dosage.

2. The method of claim 1, wherein determining a number of treatment sites, the locations of such sites, and the treatment dosage further comprises:
generating the model to include a plurality of interconnected volumes wherein each volume has one or more adjacent volumes with a shared boundary; and
conducting one or more simulations of treatment over time using the model, each simulation having a set of one or more initial parameters.

3. The method of claim 2, wherein each volume of the plurality of volumes is cuboid.

4. The method of claim 2, wherein the plurality of volumes are of equal size.

5. The method of claim 2, wherein each of the volumes includes one or more of intracellular space, extracellular space, and vascular space.

6. The method of claim 1, wherein processing the 3D imaging additionally includes obtaining one or more of tumor density, texture, and vascularity, and the model of intratumoral treatment dynamics is further based on the obtained tumor density, texture, and/or vasculature.

7. The method of claim 1, wherein the treatment is a thermal treatment and the model describes intratumoral thermal dynamics.

8. The method of claim 1, wherein the treatment is a drug treatment and the model describes pharmacodynamics.

9. The method of claim 1, wherein processing the 3D imaging of the tumor comprises segmenting the tumor from background information.

10. The method of claim 1, further comprising adjusting the determined treatment dosage(s) by a pre-determined safety margin.

11. The method of claim 1, wherein the image is obtained by retrieval from an electronic storage device.

12. A system for treatment of a tumor, comprising:
a communication interface;
a processor in communication with the communication interface, where the processor is programmed to:
obtain 3D imaging of the tumor from the communication interface;
process the 3D imaging of the tumor to obtain tumor morphology;
determine a number of treatment sites, the locations of such sites, and the treatment dosage using a model of intratumoral treatment dynamics between vascular, intracellular, and extracellular space in order for the tumor to receive a therapeutic dosage at every location of the tumor; and
provide, to a user, a treatment plan for treating the tumor at each of the determined treatment sites and with the determined treatment dosage.

13. The system of claim 12, further comprising a storage device in communication with the communication interface.

14. The system of claim 12, wherein the processor is further programmed to provide treatment instructions to an output interface.

15. A non-transitory computer-readable medium having stored thereon a computer program for instructing a computer to:
obtain 3D imaging of a tumor from a communication interface;
process the 3D imaging of the tumor to obtain tumor morphology;
determine a number of treatment sites, the locations of such sites, and the treatment dosage using a model of intratumoral treatment dynamics between vascular, intracellular, and extracellular space in order for the tumor to receive a therapeutic dosage at every location of the tumor; and
provide to an output interface, a treatment plan for treating the tumor at each of the determined treatment sites and with the determined treatment dosage.
